(19) United States
(12) Patent Application Publication
(10) Pub. No.: US 2026/0002919 A1
(43) Pub. Date: Jan. 1, 2026

(54) OPTIMAL SENSOR PLACEMENT WITH PERIMETER RESTRICTIONS FOR GASEOUS EMISSIONS

(71) Applicant: Schlumberger Technology Corporation, Sugar Land, TX (US)

(72) Inventor: Kashif Rashid, Cambridge, MA (US)

(21) Appl. No.: 18/754,237

(22) Filed: Jun. 26, 2024

(51) Int. Cl. G01N 33/00; G06F 30/28; G06F 113/08; G06F 119/02

(52) U.S. Cl. CPC G01N 33/0034; G01N 33/0047; G06F 30/28; G06F 2113/08; G06F 2119/02

(57) ABSTRACT

Embodiments presented provide for a placement methodology for monitoring a fluid. In specific embodiments, an optimal sensor placement is chosen in relation to restrictions placed upon a site during measurements from a methane source.

### CROSS-REFERENCE TO RELATED APPLICATIONS

None.

### FIELD OF THE DISCLOSURE

Aspects of the disclosure relate to monitoring of gaseous emissions. More specifically, aspects of the disclosure relate to optimal sensor placement for monitoring gaseous emissions, wherein the placement is based on perimeter restrictions.

### BACKGROUND

Gaseous emissions from commercial and industrial complexes are increasingly under scrutiny by government regulators. As governments try to minimize gaseous emissions that contribute to greenhouse gases, monitoring of those emissions is increasingly being required to be evaluated.

Monitoring of gaseous emissions is a complicated process. Sensor location is of extreme importance, otherwise the monitoring will indicate either overly conservative measurements or, conversely, extremely unreported emissions. In the case of unreported emissions, consequences can be extremely damaging for a company or an industry. Air permitting generally limits the amount of overall air pollutants that may be produced. Failure to adhere to these strict standards can lead to monetary fines that can include many millions of dollars. Such violations also tarnish a company's reputation, causing greater economic harm.

One particular problem with monitoring emissions is the locations of the sensors used to conduct the monitoring of the emissions. The locations of the sensors may, in some cases, be constrained by the overall geometry of the site. Prevailing wind conditions, meteorology, topography, the type of emission, and industrial production schedules all impact the overall ability of the industry to be able to monitor the emissions that are produced.

There is a need to provide an apparatus and methods that are easy to operate for site operators.

There is a further need to provide apparatus and methods that do not have the drawbacks discussed above related to constraints such as wind conditions, meteorology, topography, the type of emission, and industrial production schedules.

There is a still further need to reduce economic costs associated with operations and apparatus described above with conventional monitoring techniques.

There is also a need to provide a monitoring method and sensor placement scheme that is superior to conventional sensor placement technologies.

### SUMMARY

So that the manner in which the above recited features of the present disclosure can be understood in detail, a more particular description of the disclosure, briefly summarized below, may be had by reference to embodiments, some of which are illustrated in the drawings. It is to be noted that the drawings illustrate only typical embodiments of this disclosure and are; therefore, not to be considered limiting of its scope, for the disclosure may admit to other equally effective embodiments without specific recitation. Accordingly, the following summary provides just a few aspects of the description and should not be used to limit the described embodiments to a single concept.

In one example embodiment, a method to monitor emissions from a site is disclosed. The method may comprise designating a perimeter for the site to be monitored. The method may also comprise predefining locations for a number of sensors, and locations for the sensors, along the perimeter of the site. The method may also comprise defining a wind realization model for the site. The method may also comprise defining a set of candidate leak points for the site. The method may also comprise using a binary optimization method to select a first sub-set of sensor locations for evaluation of monitoring of the emissions to achieve a result. The method may also comprise further using the binary optimization method to select at least a second sub-set of sensor locations for evaluating the emissions to achieve a second result. The method may also comprise comparing the first result with the second result. The method may also comprise choosing either the first sub-set of sensor locations or the second sub-set of sensor locations based upon a factor defined by an evaluator.

In another example embodiment, a method for monitoring a leak from a leak source is disclosed. The method may comprise designating a perimeter for the site to be monitored. The method may also comprise identifying a number of sub-spaces within the perimeter in which a leak source may occur, each of the sub-spaces having a bounded region with a bounded perimeter. The method may also comprise identifying within each of the number of sub-spaces a location for a potential leak source within an interior of each sub-space. The method may also comprise locating at least one sensor to be located for monitoring each sub-space for each potential leak source, wherein the locating is done exterior to each bounded perimeter to achieve a first sub-set of sensor locations. The method may also comprise defining a wind realization model for the site. The method may also comprise calculating the center of mass of each of the subspaces to assess feasibility of each of the sensors located for monitoring. The method may also comprise using an optimization method that uses the center of mass wherein a penalty is assigned for each sensor placed within a subspace, the optimization method providing a first result. The method may also comprise further using the optimization method to select at least a second sub-set of sensor locations in a second subspace to achieve a second result, wherein the penalty is assigned for each sensor placed within the second subspace. The method may also comprise comparing the first result with the second result. The method may also comprise choosing either the first sub-set of sensor locations or the second sub-set of sensor locations based upon at least one of the first result, the second result and a factor defined by an evaluator.

In another example embodiment, a method for modeling a potential leak from a leak source at a site is disclosed. The method may comprise designating a perimeter for the site to be monitored. The method may also comprise identifying a number of sub-spaces within the perimeter in which a leak source may occur, each of the sub-spaces having a bounded region with a bounded perimeter. The method may also comprise identifying within each of the number of sub-spaces a location for a potential leak source within an interior of each sub-space. The method may also comprise locating at least one sensor to be located for monitoring each sub-space for each potential leak source, wherein the locating is done exterior to each bounded perimeter to achieve a first sub-set of sensor locations. The method may also comprise defining a wind realization model for the site. The method may also comprise defining a sensor model for the site. The method may also comprise defining a leak source model for the site. The method may also comprise calculating a wind realization model result, a sensor model result, and a leak source model result, and supplying each of the wind realization model result, sensor model result, and the leak source model result into an inversion procedure. The method may also comprise using the inversion procedure, developing a detection coverage map for the site.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the drawings. It is to be noted; however, that the appended drawings illustrate only typical embodiments of this disclosure and are therefore not be considered limiting of its scope, for the disclosure may admit to other equally effective embodiments.

FIG. 1 is a plot plan of an emission site showing a general site definition.

FIG. 2 is a plot plan of an interior feasible sensor placement case for FIG. 1.

FIG. 3 is a plot plan of an exterior feasible sensor placement case.

FIGS. 4A and 4B show perimeter placement of sensors.

FIGS. 5A through 5D show a perimeter location filter by record count.

FIG. 6 is a plot of a master site.

FIG. 7 is an example of defined sub-spaces for the master site.

FIG. 8 is an example of definition classes.

FIG. 9 is a schema diagram for FIG. 6.

FIG. 10 is a depiction of defining a convex set where both feasible interior and exterior alternatives are present.

FIG. 11 is a set of three plots using a coverage evaluation procedure in one example embodiment of the disclosure.

FIG. 12 is a methodology for developing a coverage map in one example embodiment of the disclosure.

FIG. 13 is a map of optimal sensor placement in one example embodiment of the disclosure

FIG. 14 is a graph of scaled response versus distance as a penalty measure for infeasibility.

FIG. 15 is a graph of factors and evaluation steps for sensor placement evaluation.

FIG. 16 is a graph of optimal placement results for 6 sensors.

FIG. 17 is a graph of optimal placement results for 6 sensors.

FIG. 18 is a graph of an established synthetic wind model.

FIG. 19 depicts graphs of wind-sensor data from a synthetic wind model for two given sensors.

To facilitate understanding, identical reference numerals have been used, where possible, to designate identical elements that are common to the figures (“FIGS”). It is contemplated that elements disclosed in one embodiment may be beneficially utilized on other embodiments without specific recitation.

### DETAILED DESCRIPTION

In the following, reference is made to embodiments of the disclosure. It should be understood; however, that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the following aspects, features, embodiments, and advantages are merely illustrative and are not considered elements or limitations of the claims except where explicitly recited in a claim. Likewise, reference to “the disclosure” shall not be construed as a generalization of inventive subject matter disclosed herein and should not be considered to be an element or limitation of the claims except where explicitly recited in a claim.

Although the terms first, second, third, etc., may be used herein to describe various elements, components, regions, layers, and/or sections, these elements, components, regions, layers and/or sections should not be limited by these terms. These terms may be only used to distinguish one element, components, region, layer, or section from another region, layer, or section. Terms such as “first”, “second”, and other numerical terms, when used herein, do not imply a sequence or order unless clearly indicated by the context. Thus, a first element, component, region, layer, or section discussed herein could be termed a second element, component, region, layer, or section without departing from the teachings of the example embodiments.

When an element or layer is referred to as being “on”, “engaged to”, “connected to”, or “coupled to” another element or layer, it may be directly on, engaged, connected, or coupled to the other element or layer, or interleaving elements or layers may be present. In contrast, when an element is referred to as being “directly on”, “directly engaged to”, “directly connected to,”, or “directly coupled to” another element or layer, there may be no interleaving elements or layers present. Other words used to describe the relationship between elements should be interpreted in a like fashion. As used herein, the term “and/or” includes any and all combinations of one or more of the associated listed terms.

Some embodiments will now be described with reference to the figures. Like elements in the various figures will be referenced with like numbers for consistency. In the following description, numerous details are set forth to provide an understanding of various embodiments and/or features. It will be understood; however, by those skilled in the art, that some embodiments may be practiced without many of these details, and that numerous variations or modifications from the described embodiments are possible. As used herein, the terms “above” and “below”, “up” and “down”, “upper” and “lower”, “upwardly” and “downwardly”, and other like terms indicating relative positions above or below a given point are used in this description to more clearly describe certain embodiments.

Aspects of methods described may be included onto a non-volatile memory system. For definitional purposes, a non-volatile memory system may be a memory system that does not wipe clean after termination of electrical power to the system. Examples of non-volatile memory systems may be compact disks, solid-state drives, and universal serial bus devices. These memory systems may be used to store program executable method steps for a computer, server, or computing arrangement.

Embodiments of the disclosure provide selecting a number and location of sensors for monitoring a gaseous stream. In non-limiting embodiments, methane may be monitored at a remote site. The placement of the sensors is chosen to maximize the coverage and sensitivity of readings so that the owner of the facility may accurately identify the effluents from the facility. Although discussed as measuring methane, other effluents may be measured using the methodologies disclosed.

Several different solutions to the problem of accurately locating sensors are presented. Such solutions include, A) Interior Feasible Methods, B) Exterior Feasible Methods, C) Geometric Design Methods, D) Normalized Variable Methods, E) Uniform Edge Variable Methods and F) Binary Selection Methods. Each of the methods will be discussed below.

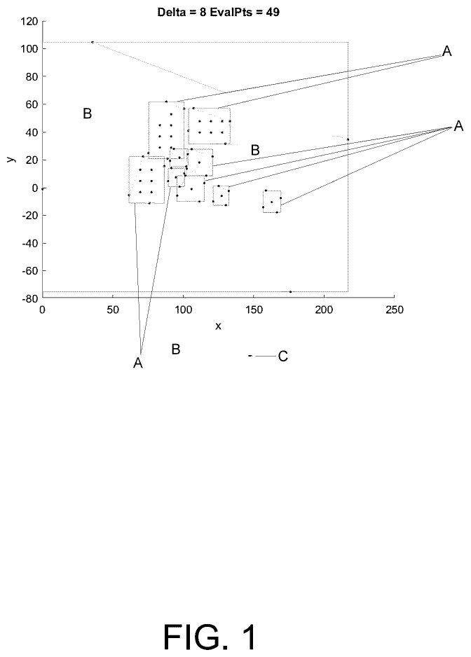

The methods described have different levels of complexity as well as associated difficulty in realizing an optimal solution. FIG. 1 shows a general site layout example. A plot may be generated of the overall site plan. As illustrated, in x and y coordinates, the outline of a site that may produce methane emissions is illustrated. In some instances, the site may be a hydrocarbon production facility, such as an oilfield. In this embodiment, the outline of the property is noted on the plan.

Box A defines the outer bounds of the site indicating the search domain. Nine sub-spaces are defined inside the site bounds. Each is marked by lines A and B. Points C mark the set of potential leak sources that collectively are used to evaluate the coverage measure for a given set of sensors on the site, along with the prevailing wind conditions.

The objective of the method is to identify the number and placement of sensors to maximize the measurement coverage area as well as the quality of the measurements. In this embodiment, the sensor placements are not permitted in the sub-spaces; so these sub-spaces serve as restricted zones. Other restricted zones may be added by need (to mark roads or other limitations) but are not present in this example.

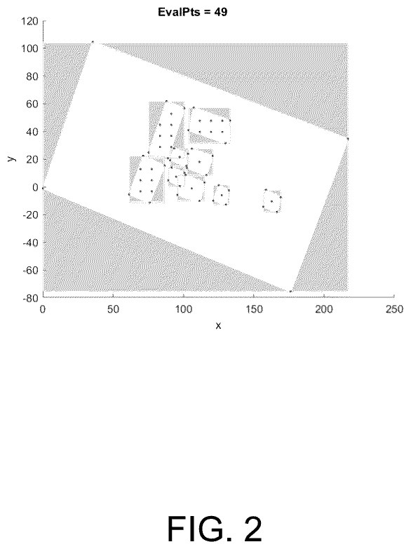

Referring to FIG. 2, the results of the interior feasibility method of FIG. 1 are illustrated. In this embodiment, sensors may be placed on the interior of the site and may approach the perimeter as needed. In this method, the definition of sub-spaces is performed and restricted zones are established as described above. As will be understood, restricted zones may be added or deleted according to the geometries and details of the area.

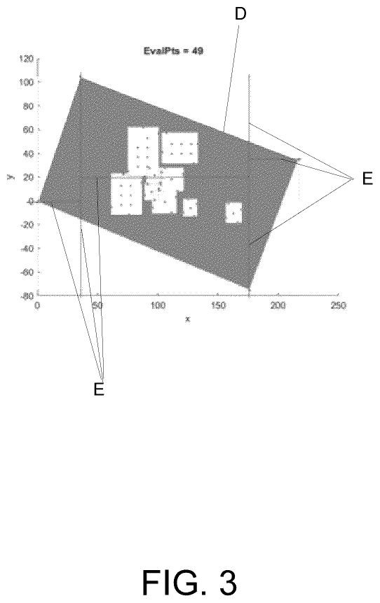

Referring to FIG. 3, a site is shown where an exterior feasible method may be used. In this embodiment, sensors may be placed outside of the site, noted by box D. This may be performed to maximize the coverage measurement capability. The sub-spaces, noted by the rectangular boxes, are contained in an infeasible area (area that is excluded for placement of sensors). The stipulation of placement feasibility entails partitioning the site area into restricted zones (see imposed linear cuts shown as lines E) that ensure placement feasibility. Hence, feasibility is defined by the 6 restricted zones in the example above. Each site is thus suitably partitioned.

For geometric designs, a method may be used to place sensors according to a pattern. This method automatically generates a uniformly distributed set of sensors arranged along the perimeter line. Infeasible sensors are filtered from the design so that each design is feasible. In this method, there is no requirement for sub-spaces definition. The design for a given number of sensors is deterministic and each can be evaluated in parallel, giving a coverage estimate with an increasing number of sensors. Notably, the solutions for a small number of sensors may not be optimal, while a greater number of sensors will give a saturated measure as an upper bound for the coverage possible. One limitation of the geometric design stems from the definition of the perimeter. If the perimeter extends to regions far away from the source locations, an artificial perimeter line is recommended on the interior, or a restriction zone should be added, to prevent placement in undesirable regions for oversaturation.

In the geometric design methodology described above, the problem becomes computationally more involved with an increasing number of sensors. One option is to provide a variable that defines the number of sensors along a given edge of the perimeter line. Thus, the number of variables is reduced to the number of edges in the perimeter definition. Each edge variable provisions a uniformly distributed set of sensors. Collectively these are used to evaluate the coverage measure. There is no need for explicit sub-space stipulation, but restricted zones are required to penalize poor placement. The fact that each design results in a varying number of sensors dictates special handling of constraint sets (for example, those that assert separation distance requirements). This ensures that the solver manages a consistent problem with respect to the size of sensors.

For the normalized perimeter variables method, an optimal placement of sensors along a perimeter is sought. Each sensor in the search provides one variable that is normalized over the length of the perimeter. Thus, the solver establishes the location of each sensor to maximize the coverage measure as a nonlinear problem. Sub-spaces are not required as placement is only permitted on the perimeter, but restricted zones are necessary to penalize misplacement of sensors in poor locales.

In a pre-defined binary selection method, the definition of sensors along the perimeter line subject to some minimum separation distance is performed. In this method, a larger number of sensors may be required; however, it is intended that a data extraction and record generation procedure is applied to each sensor in a pre-processing step. This procedure yields a coverage map (based on record count) at each point along the perimeter; indicating the potential utility of the placement of a sensor at that point, given the prevailing wind conditions and is not limited to only perimeter placements, as discussed below.

Subsequently, a binary optimization problem can be defined to pick a sub-set of sensor locations based on the notion of maximizing the information provisioned by the selected sub-set subject to minimum separation distance conditions. As the problem is based on the record count as a proxy to the inversion quality (with the assumption that the greater the number of records available, the better the inversion result) the computational cost is limited to the binary optimization method used to select the sub-set of desirable locations. The objective evaluation cost is minimal, so the algorithm efficiency dictates performance. Advanced meta-heuristics can be used to manage the binary nonlinear optimization problem with the outcome of a selection of desirable locations. In some embodiments, a Monte Carlo method may be used for selection.

In a final step, another binary optimization problem may be defined. An effective number of sensors may be picked over the pre-defined and pre-selected set of locations, in which each objective evaluation involves a collection of full inversion solutions. In some embodiments, the sample points used for the evaluation measure and the location of sensors can also vary in height, e.g. (consider 2 points with differing heights in meters [x y 5] and [x y 10]. A metric may be generated that is the quality of the coverage achieved with the given number of sensors and their stipulated locations. This procedure can be computationally intensive for large numbers of retained sensors; however, some expedient approaches to tackle this problem (with an expensive objective measure) include the use of a Monte-Carlo method, full enumeration of the set of designs for a small number of sensors, or to use a meta-heuristic suitable for binary nonlinear optimization (e.g. a Tabu Search method). A solution will indicate the number and location of sensors necessary to optimize the coverage measure over the given site.

Note that the use of simple geometric patterns does not result in an optimal design. This is particularly true when the number of sensors is small. Thus, there is a need to optimize the placement of sensor locations over the perimeter in an efficient manner, and the method described herein is one means to achieve this result. This method offers a greater utility to treat a much broader set of problems in that the pre-defined locations of the sensors need not be limited to points along the perimeter. The sensor locations may be distributed over the entire feasible domain of the site by need, and the records generated at each point will provision a proxy coverage map that can be used to select the potential locations (as indicated above when restricted to the perimeter). A similar strategy of selecting the desired number of sensors at given locations can be used when the sensor placement is not restricted only to the perimeter. In embodiments, the location of sensors can be pre-defined along the perimeter. In other embodiments, the location of sensors may be placed along the perimeter and other locations as required.

In the aforementioned methods, a single objective entails multiple inversion evaluations given the number of candidate leak points, the number of sensors, and the number of wind realizations used. Thus, the coverage evaluation method can be computationally involved. The entire process is repeated for a design that may differ only slightly. In one example embodiment, a design with 10 sensors may have previously been evaluated and a new design is sought in which only one sensor is marginally displaced. In this instance, the objective measure will be calculated from the start. To overcome this computational inefficiency, the location of sensors can be pre-defined along the perimeter and all the data can be generated and stored in a pre-processing step. That is, the record set at each possible sensor location can be established in advance and used directly when that location is selected for evaluation purposes. This reduces the computational cost associated with similar designs, and further speeds up the overall coverage evaluation cost by resorting to the stored data. To this end, sensors can be placed along the perimeter in close proximity and the data established at each location offline.

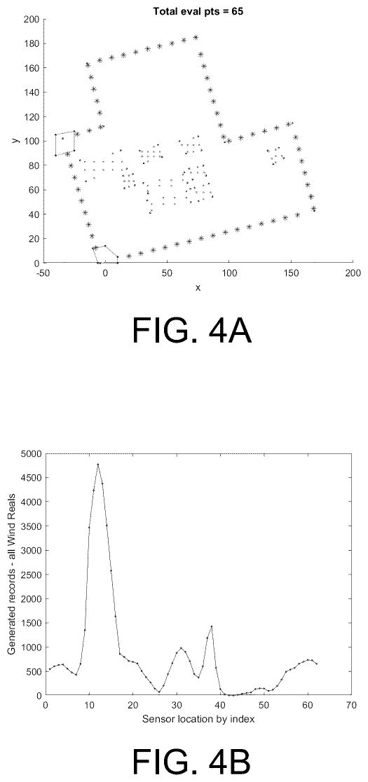

For the pre-defined binary selection method, FIG. 4A shows a site with an abundant number of sensors at fixed locations. FIG. 4B shows the associated number of records that are generated at each location along the perimeter by index (given the set of wind realizations and the time-period of interest, say 24 hours). In this embodiment, some locations provide many records, while others do not. The locations that do not provide many records are considered less favorable.

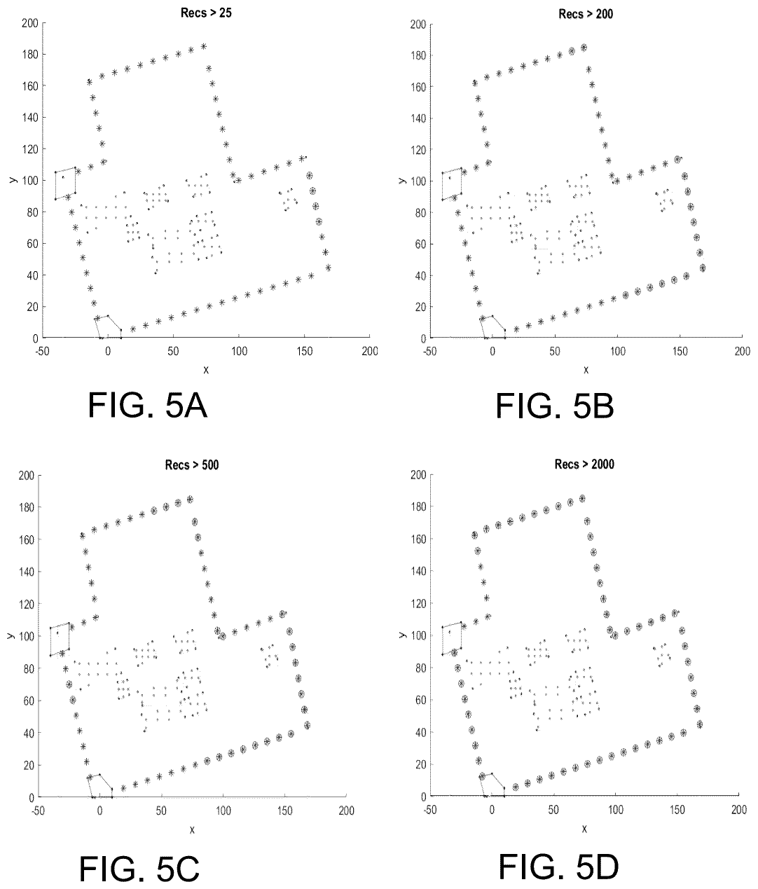

For the pre-defined binary selection method, in FIG. 5, the sensor locations are filtered based on the number of records generated. The green markers indicate the acceptable location set in each case, for imposed limits of 25, 200, 500, and 2000 records. In some instances, a color plot is more representative. Using filters, or a more involved selection scheme, the set of desirable sensor locations (subject to minimum separation distance) can be selected for consideration. One such scheme is based on a “greedy” procedure wherein “greedy” is defined as selecting from best to worst, subject to minimum separation distance requirement. As noted above, the record count may be considered a proxy to full inversion.

In a subsequent step, the full coverage measure may be evaluated to obtain the optimal number and selected location of sensors. Note, that while the data is predefined at each location, the coverage measure still entails numerous inversion evaluations. These evaluations can be undertaken in parallel if that provision is made available. The search method may involve full enumeration over a small set of selected sensors, employ a Monte-Carlo scheme, or use a meta-heuristic for binary nonlinear optimization, in non-limiting embodiments. A quantum computing setup may also be used to solve this problem, which concerns picking n sensors from N informed locations, to maximize the information available (e.g., the total record count).

This method affords greater flexibility and utility as compared to some of the other perimeter restricted approaches, given that the set of sensor locations need not be limited to the perimeter line. The sample heights can also be made to vary as needed. The samples may be stipulated over the feasible interior or exterior by need. This eliminates the extensive and complex arrangements for sub-space and restricted zone stipulation as per the first two methods. In addition, a coverage map of sensor placement utility can be furnished over the set of samples over the site and used for selection purposes for the perimeter restricted case.

Aspects of the disclosure also concern both optimal sensor placement and optimal inversion subject to sub-space feasibility. In these embodiments, a site with specified bounded sub-regions indicative of equipment groups, enclosed areas, buildings, and other designated regions, may be deemed as either locations of potential leak sources in the inversion problem or locations that define restricted zones in the sensor placement problem. In these aspects, the original sensor placement and leak optimization procedure to accommodate a fifth dimension (representing an index to a given sub-space) can help localize leaks to particular zones and ensure sensor placement subject to site restrictions (the excluded regions).

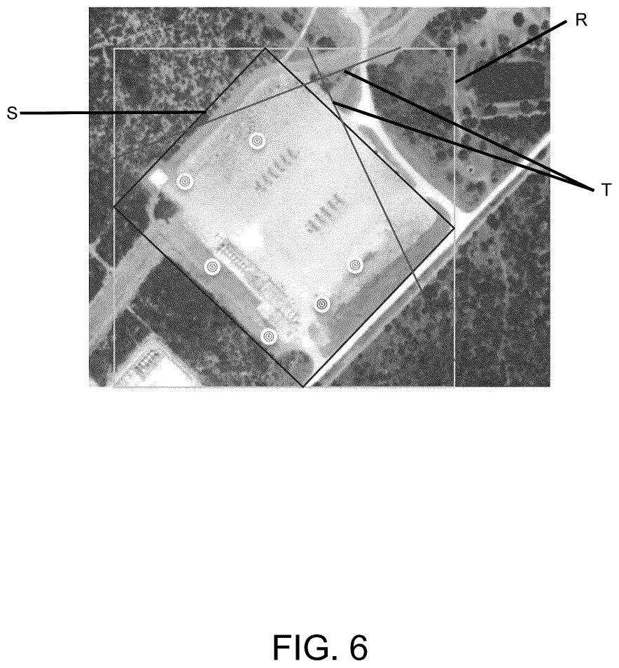

FIG. 6 shows an example site for continuous leak monitoring. The master problem is defined with one sub-space that includes the global search bounds “R” along with any additional site restrictions in the form of linear cuts (the lines labeled S and T. For the placement problem portion of the solution, the sensors can be located with respect to site feasibility. For the source inversion portion of the solution, the leak is sought on the interior of the defined feasible space if constrained, or over the bounds if unconstrained.

Sub-spaces are graphically noted in FIG. 6. The first sub-space indicates the master problem in which the source inversion is not subject to sub-space restrictions.

Site example defined with one sub-space (nb=1) representative of the master problem are illustrated in FIG. 6. The search bounds (labeled R) with linear constraints (lines S and T) are illustrated for clarity. The circles marked in FIG. 6 indicate the location of sensors.

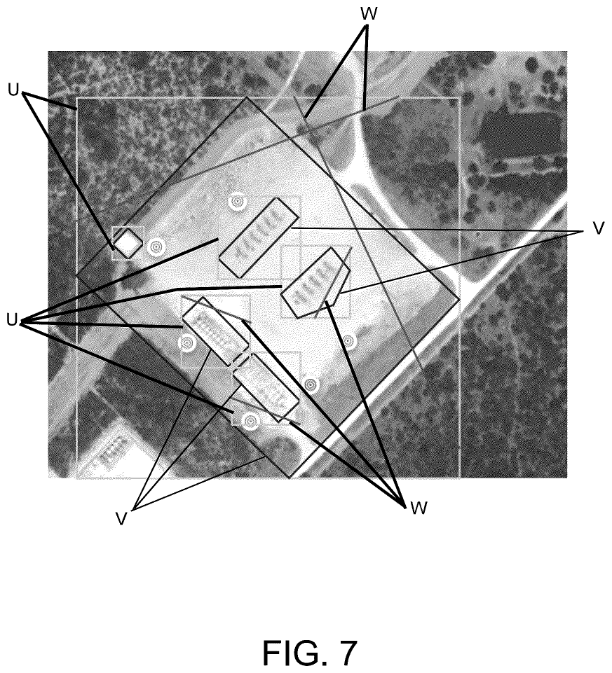

FIG. 7 shows an example site with six (6) defined sub-spaces. The first sub-space defines the master problem, as noted above. Five additional sub-spaces are shown. The additional 5 sub-spaces identify regions in which leak sources may occur (equipment groups or zones), and in which a sensor is not permitted to be placed. Each sub-space comprises a bounded region (labeled U) and a set of linear constraints (labeled V and W) that mark possible feasibility. Thus, for a given sub-space, a leak source is specified by a feasible interior, while sensor placement is dictated by a feasible exterior. The center of mass of the points is used to assert feasibility over each sub-space as required. Given site information, the sub-spaces can be defined manually (in 2D or 3D) or crafted automatically using a segmentation procedure. Subsequently, optimal sensor placement or source inversion can be performed in accordance with the feasibility asserted by the sub-spaces. The mathematical formulation is defined as recited below.

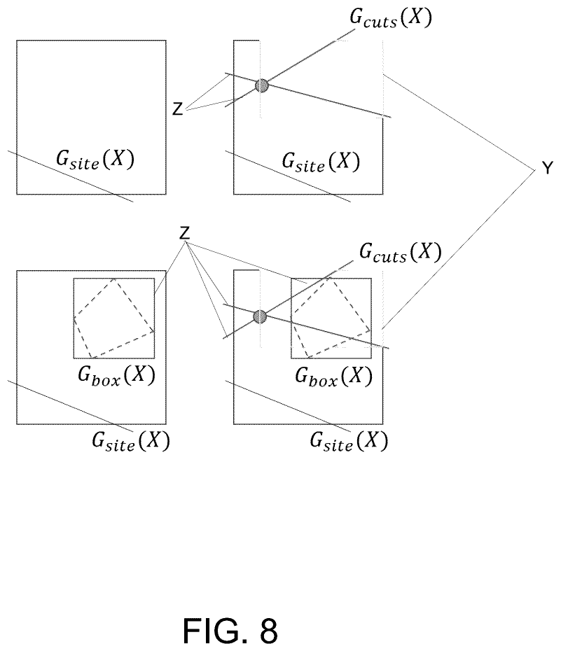

FIG. 8 shows the schema for four evaluation cases (as applied to either sensor placement problem or the source inversion problem): In case 1 (noted in the top left), the search is over the master bounds subject to site constraints only. In case 2 (noted in the top right), details are as illustrated per case 1, but additional linear cuts (labeled Z) are imposed along with reduced bounds (labeled Y). In case 3 (noted in the bottom left), as per case1, the search is over the master bounds subject to site constraints, but the conditions imposed by each sub-space are also imposed. That is, given sub-space bounds and linear constraints. In case 4 (noted in the bottom right) the disclosure is the same as case 3, but there is an inclusion of linear cuts and reduced bounds.

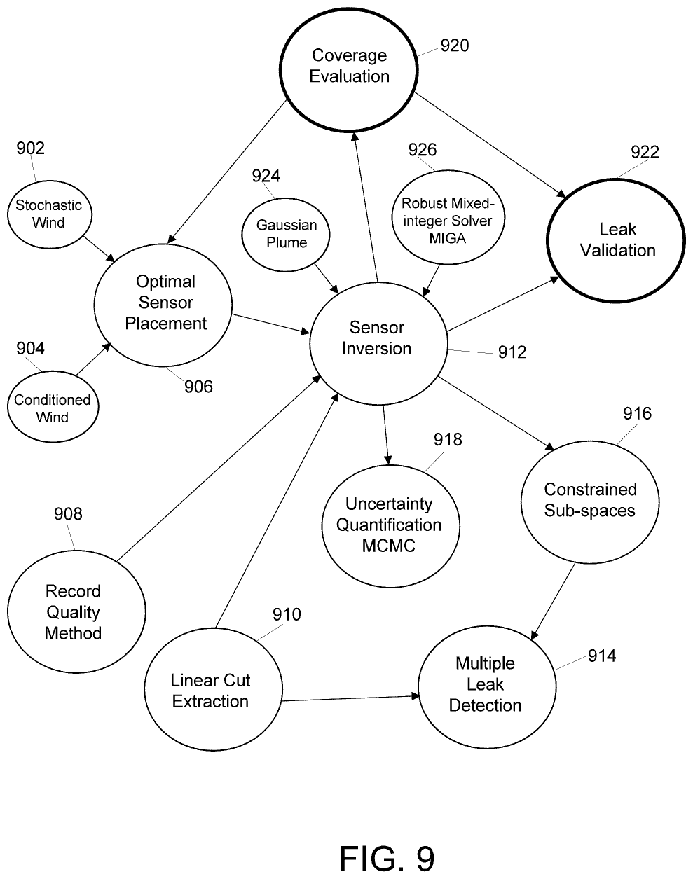

FIG. 9 shows a typical solution landscape for embodiments of the disclosure. As illustrated, a stochastic wind model 902 and a conditioned wind model 904 are used to conduct an optimal sensor placement 906. A record quality method 908 is used to verify and ensure data quality. The data may be from a database or may come directly from sensors located around the evaluation site. A linear cut extraction method 910 may be used for the site that feeds data into a calculation program for sensor inversion 912 as well as being used for multiple leak detection 914. Output from the sensor inversion 912 may be used in conjunction with constrained sub-spaces 916 to aid in potential multiple leak detection 914.

Sensor inversion 912 may also be used to ascertain uncertainty quantification 918, coverage evaluation 920, and leak validation 922. Differing mathematical solutions may be used to help in the solution inversion. In the illustrated embodiment, a Gaussian plume solver 924 may be input into the sensor inversion 912 for solution generation. Other types of plume mathematical evaluations may be used and as such, a Gaussian plume solver 924 may be one possible alternative. In addition to the above, a robust mixed-integer solver 926 may also be used by the sensor inversion 912.

As input, an image of a site that may contain a methane leak, or desire methane sensor coverage, is required. A local coordinate system is determined. Site bounds are then established through the local coordinate system. Other site constraints are established. For both the site bounds and site constraints, the coordinates may be entered as a two-dimensional location.

Equipment group locations, in either two or three dimensions may be located within sub-spaces. Feasible sensor placements within the site bounds may also be located. Restrictions or other limitations upon the site may also be input into the model. From the output, an output may be generated.

In the below paragraph, a master problem is discussed where the number of sub-spaces is one. Discussion of possible mathematical solutions to the steps recited above are discussed. A problem definition may be established where site BOX properties are set. A number of sub-spaces is defined as the value n_{b} where n_{b}=1→no sub-spaces and:
b∈[1n_{b}]
BOX(b).LB[1 4]
BOX(b).UB[1 4]
BOX(b).GBOX[n_{g}6]
BOX(b).SAMP[sample set]
BOX(b).PSET[convex set]
BOX(b).GP2 [ng 3] [x y 1]
BOX(b).GP4 [ng 5] [x y z r 1]
BOX(b).GP5 [ng 6] [x y z r b 1]
BOX(b) EPTS (leak evaluation set)

An additional problem definition for value n_{b} is provided. In this problem definition, the number of sub-spaces is defined as the value n_{b} (where n)=1→no sub-spaces case) to yield information for each subspace as follows:
b∈[1 n_{b}]
BOX(b).LB[1 4]
BOX(b).UB[1 4]
BOX(b).GBOX[n_{g} 6]
BOX(b).SAMP[sample set]
BOX(b).PSET[convex set]
BOX(b).GP2 [ng 3] [x y 1]
BOX(b).GP4 [ng 5] [x y z r 1]
BOX(b).GP5 [ng 6] [x y z r b 1]
BOX(b) EPTS (leak evaluation set)

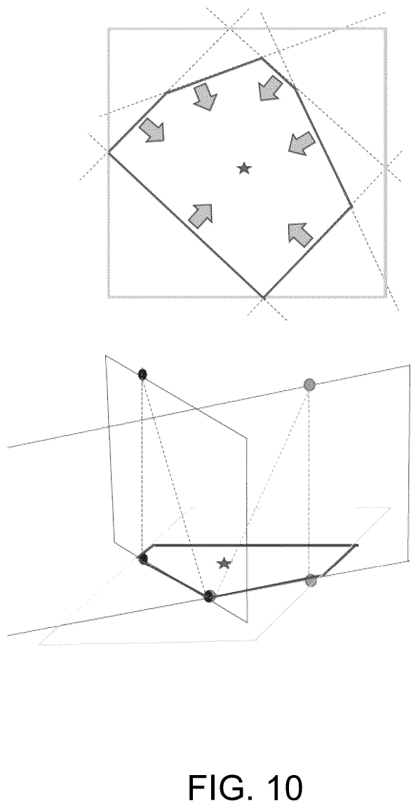

In the above, a collection of samples may define a subspace and a convex set (perimeter) is established for each. A set of linear constraints are extracted from the convex set of points and a center of mass technique may be used to indicate the feasibility of sensor placement. In embodiments, leak isolation is determined by a feasible interior location, while sensor placement is possible or feasible on the exterior of a subspace per FIG. 10.

Given a set G (X) that is compactly evaluated: AX̅≤0, with X̅=[x y z r b 1]^{T}. Per FIG. 8, the problem classification is as follows: for the top row: sub-space [FALSE]: Linear Cuts False and Linear Cuts True, and for the bottom row: sub-space [TRUE]: Linear Cuts False and Linear Cuts True.

For the sensor inversion procedure described above, the inputs may include wind, sensor data, solar data, sensor location, or site information BOX. Data is extracted over a desired time period, T. Generated records, such as data that occurs within specified time windows, t, may be input. Extracted linear cuts using all data in the desired time period T may also be used as an input. Updated record quality measurements may also be input.

The inversion may be performed and uncertainty estimations may be generated. The output may be in the form of: Xopt=[x y z r b] and distributions. Per FIG. 8:

$$min F ( X ) = \sqrt{\frac{1}{R} {\sum }_{i = 1}^{R} {( {M}_{i}^{obs} - {M}_{i}^{pred} ( X \vert W , U ) )}^{2}}$$

$${G}_{site} ( X ) \leq 0$$

$${G}_{cuts} ( X ) \leq 0$$

$${x}_{L}^{i} \leq {x}_{l}^{i} \leq {x}^{i} \leq {x}_{u}^{i} \leq {x}_{U}^{i}$$

$${x}^{i} ∈ ℝ i ∈ [ 14 ]$$

$$X = [ x y z r ]$$

$$with the following definitions :$$

$${M}^{pred} = plume ( X \vert W , U )$$

$$X = [ {source}_{x} {source}_{y} {source}_{z} {source}_{r} ]$$

$$Y = [ {wind}_{air} {wind}_{speed} {wind}_{stability} ]$$

$$U = [ {sensor}_{x} {sensor}_{y} {sensor}_{z} ]$$

$${{REC}_{i} [ W U {M}^{obs} ]}_{i} .$$

The problem may be solved where:

$$min P ( V )$$

$$s . t . {G}_{site} ( V ) \leq 0$$

$${G}_{cuts} ( V ) \leq 0$$

$$V = [ {v}_{x} {v}_{y} {v}_{z} {v}_{r} b ]$$

$${v}_{L}^{i} \leq {v}^{i} \leq {v}_{U}^{i}$$

$${v}^{i} ∈ ℝ i ∈ 14 ]$$

$${v}^{r} ∈ ℝ or ℤ ∈ [ 1 {n}_{b} ]$$

$$where :$$

$$X = f ( V ) = [ x y z r ] and V = [ {v}_{x} {v}_{y} {v}_{z} {v}_{r} b ]$$

$$P ( V ) = F ( X ) + γ \sum max {( 0 , {g}_{box} ( X ) )}^{2}$$

$$F ( X ) = \sqrt{\frac{1}{R} {\sum }_{i = 1}^{R} {( {M}_{i}^{obs} - {M}_{i}^{pred} ( X \vert W , U ) )}^{2}}$$

$$b = {V}^{5} or round ( {V}^{5} )$$

$$BLB = BOX ( b ) . LB$$

$$BUB = BOX ( b ) . UB$$

$${G}_{box} = BOX ( b ) . GBOX$$

$$GLB = BOX ( 1 ) . LB$$

$$HUB = BOX ( 1 ) . UB$$

$$For i ∈ [ 14 ]$$

$${v}^{i} = V ( i )$$

$${x}_{min}^{i} = GLB ( i )$$

$${x}_{max}^{i} = GUB ( i )$$

$${x}_{L}^{i} = BLB ( i )$$

$${x}_{U}^{i} = BUB ( i )$$

$${x}_{i} = ( \frac{{v}^{i} - {x}_{min}^{i}}{{x}_{max}^{i} - {x}_{min}^{i}} ) ( {x}_{U}^{i} - {x}_{L}^{i} ) + {x}_{L}^{i}$$

In embodiments, let wᵢ represent the weight of the i-th record in array W of size [R, 1] where the weights wᵢ are defined such that

$${\sum }_{i = 1}^{R} {w}_{i} = 1.$$

The weighted error term E (X) is defined:

$$F ( X ) = \sqrt{{\sum }_{i = 1}^{R} {{w}_{i} ( {y}_{i} - {R}_{i} )}^{2}}$$

Where, yᵢ is the i-th model response, Rᵢ is the i-th record observation and wᵢ is the i-th record weight ∈[0 1]. If each record has an assigned quality measure qᵢ defined in the array Q of size [R, 1], the associated weight is

$${w}_{i} = \frac{{q}_{i}}{\sum Q} ,$$

if the weights are uniformly assigned, with

$${w}_{i} = \frac{1}{R} ,$$

then the function above is equivalent to a root mean square error measurement.

### Coverage Evaluation Procedure

One example coverage evaluation procedure is described below:
Wind and Solar data are obtained for a period of interest, T:
An evaluation set E=[nₑ 2]−uniform grid, over sub-spaces, etc.
The values z and r are defined and fixed.
Set i-th leak, Sₗₑₐₖ [x y z r]
For each leak point:
Solve the Sensor Inversion Problem->Xₒₚₜ
Establish optimality gap: ∥Xₒₚₜ Sₗₑₐₖ∥→dX₂ dX₃ dR dX̅₄
Set selected optimality gap measure: D_{gap}
Given optimality gap; define a solution as good, medium, or poor.
Increment solution counter for good, some, or poor coverage.
Set the selected metric: % good, % medium, % poor, % some (good+medium)
Define Coverage Measure: C(U|W,E) where U=[uₓ u_{y} uₓ u_{y}][1 2 nₛ]
and E=[nₑ 2].
Given the set of Wind Realizations i∈[1 n_{w}]
Given the set of evaluation points E→Collection BOX(b).EPTS
Evaluate the Coverage Measure C(U|Wᵢ,E)
Plot the Coverage map for good, medium, some, or poor locations, as needed.
Define the collective measure

$$\overline{C} = \frac{1}{{n}_{w}} {\sum }_{i = 1}^{{n}_{w}} C ( U \vert {W}_{i} , E ) .$$

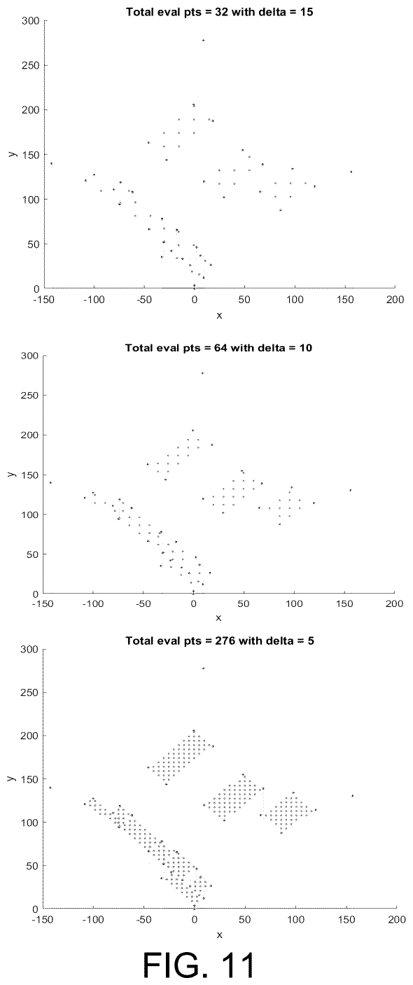

As represented by FIG. 11, three sample plots of a hypothetical coverage evaluation procedure are presented, with increasing density of candidate samples given by 32, 64 and 276 points.

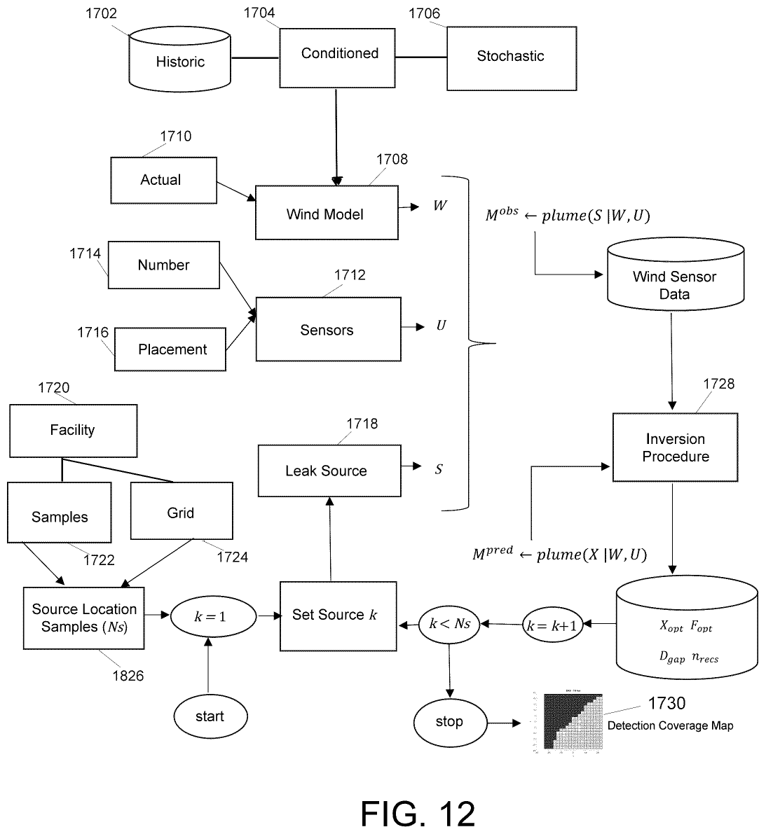

Referring to FIG. 12, a coverage map method is illustrated. In embodiments, historic data 1702, conditioned data 1704, and stochastic methods 1706 may be used to input data into a wind model 1708. Actual data from a prospective site may also be used at 1710. A sensor model 1712 is also created that will take a number 1714 and placement 1716 of sensors to create a numerical output.

A leak source 1718 may also be defined as S and may take data from, for example, a facility 1720, samples 1722, a grid 1724, source location samples 1726, as well as output from an inversion procedure 1728 to define the leak source 1718. Each of the wind model 1708, sensors 1712, leak source 1718 may be input into an inversion procedure 1728 for output that may be illustrated in a detection coverage map 1730.

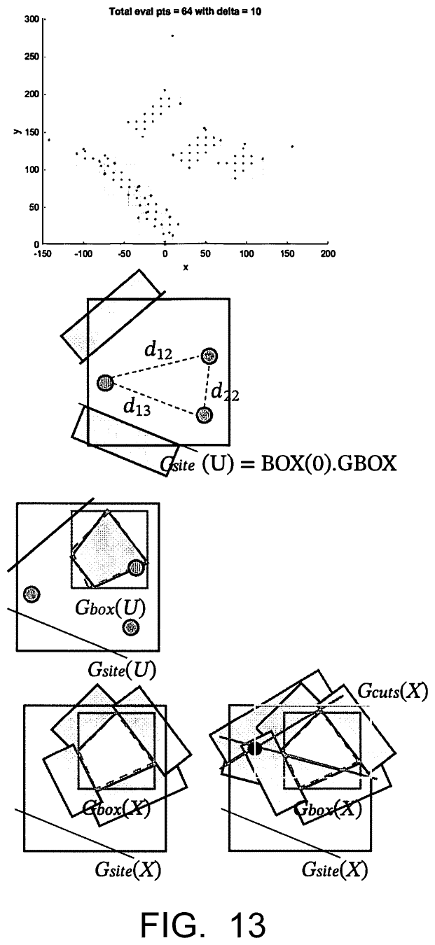

A method for optimal sensor placement may also be achieved. Referring to FIG. 13, a graphical depiction of the placement may be seen as well as boundary limitations. For this placement, the optimal placement for a set of sensors (nₛ)

$$U = [ {u}_{x}^{1} {u}_{y}^{1} … {u}_{x}^{{n}_{s}} {u}_{y}^{{n}_{s}} ]$$

With n_{w} wind realizations:

$$W = [ {W}_{1} … {W}_{{n}_{w}} ]$$

and set of evaluation points:

$$E = [ {E}_{2} … {E}_{{n}_{b}} ]$$

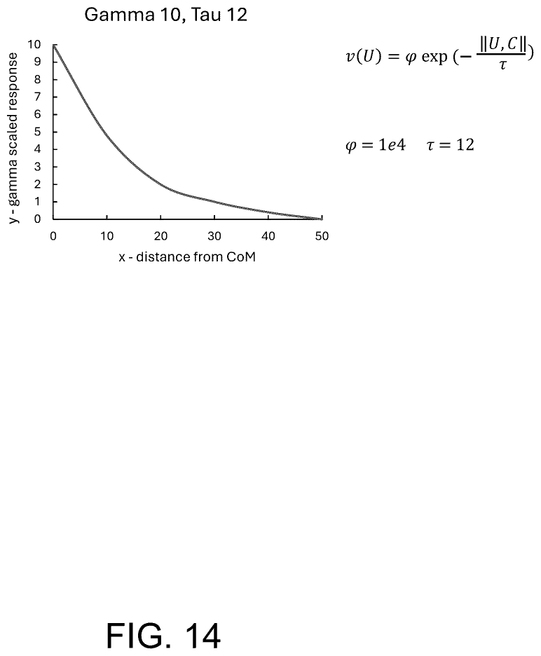

For Optimal Sensor Placement, referring to FIG. 14, the following problem is solved:

$$arg max S ( U \vert W , E ) = \overline{C} ( U \vert W , E ) - {P}_{site} ( U ) - {P}_{sub} ( U ) - {P}_{zone} ( U )$$

$$s . t . {G}_{site} ( U ) \leq 0$$

$${G}_{sep} ( U ) \geq {d}_{min}$$

$$\overline{C} = \frac{1}{{n}_{w}} {\sum }_{i = 1}^{{n}_{w}} C ( U \vert {W}_{i} , E )$$

$$U = [ {u}_{x}^{1} {u}_{y}^{1} … {u}_{x}^{{n}_{s}} {u}_{y}^{{n}_{s}} ]$$

$${u}_{L}^{j} \leq {u}_{}^{j} \leq {u}_{U}^{j} j is odd$$

$${u}_{L}^{k} \leq {u}_{}^{k} \leq {u}_{U}^{k} k is even$$

$${u}^{i} ∈ ℝ and i ∈ [ 12 {n}_{s} ]$$

$$Where :$$

$${P}_{site} = {\sum }_{i = 1}^{{n}_{s}} {\sum }_{j = 1}^{{n}_{g}} γ {max ( 0 , {g}_{i} ( \overline{u} ) )}^{2}$$

$${P}_{sub} = {\sum }_{i = 1}^{{n}_{s}} {\sum }_{k = 1}^{{n}_{b} - 1} γ max ( 0 , {v ( ( \frac{Ui}{Ck} ) )}^{2}$$

$${P}_{zone} = {\sum }_{i = 1}^{{n}_{s}} {\sum }_{j = 1}^{{n}_{g}} γ max ( 0 , {v ( ( \frac{Ui}{Cj} ) )}^{2}$$

$$i = [ {u}_{x}^{i} {u}_{i}^{j} ] i ∈ [ 1 {n}_{s} ]$$

$$v ( \frac{Ui}{Cj} ) = ϕ exp ( - \frac{‖ Ui , Cj ‖}{τ} )$$

$$γ = 1 e 5 , ϕ = 1 e 4 , τ = 12.$$

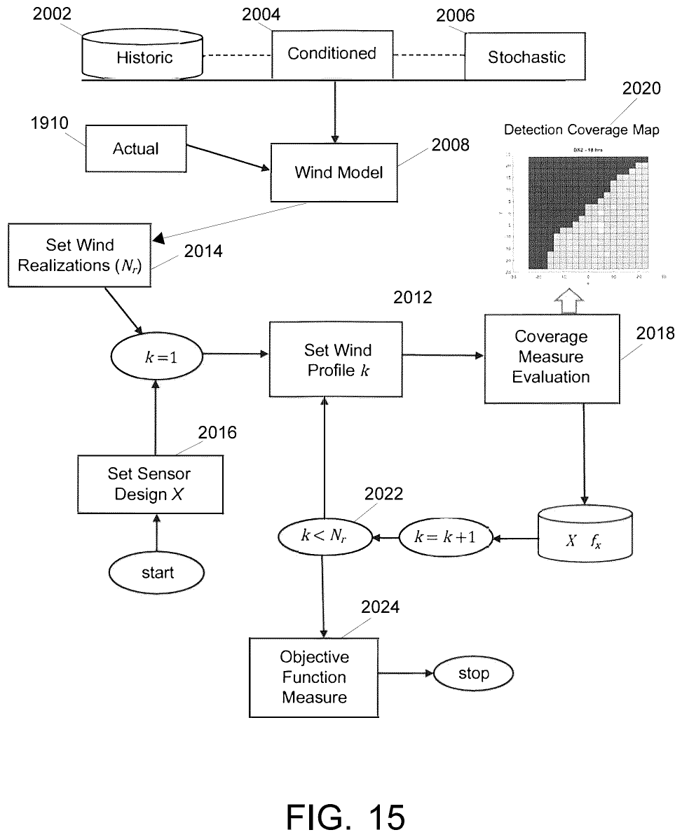

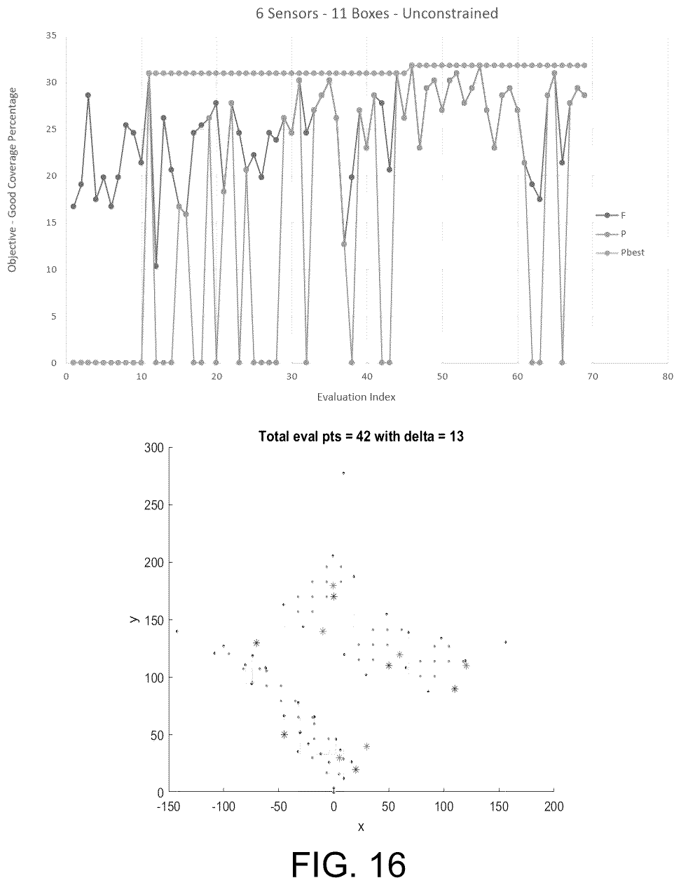

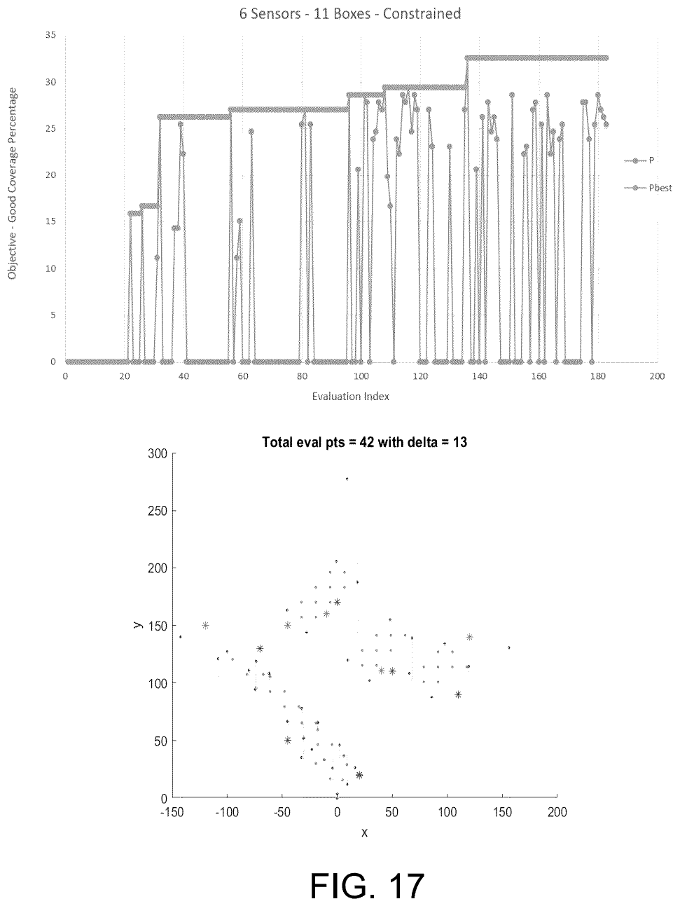

Referring to FIG. 15, for the methodology for sensor placement for evaluations, conditioned data 2004 based on historic data 2002 or stochastic data/methods 2006 is given as an input to the wind model 2008. Actual data 1910 from a sensor site may also be used. The output of the wind model 1908 may be fed into setting a wind profile k 2012. Other inputs may be provided to the wind profile 2012 such as setting wind realizations 2014, and sensor designs 2016. A feedback loop also provides data from a coverage measure evaluation 2018. The results from the coverage measure evaluation 2018 and the detection coverage map 2020 and integer increase k 2022 may be fed into an objective function measure 2024. FIGS. 16 and 17 provide sample results. The coverage measure 2018 is established for wind realization k. The value k is then incremented. If k≤N, then evaluate the next wind realization coverage value. If k=Nᵣ 2022, use the data to establish the objective measure; defined here as the mean coverage measure. An alternative risk-weighted value can be used as required. The objective value is returned as the output in 2024.

A synthetic wind model is also created with two inter-twined random walks. A time period (T=24 h) and frequency (sec or min). Wind direction may be set between [0 360] deg. Wind speed may be set between [1 15] m/s. The initial wind direction and speed may be also specified as well an intra-block period (t=20 min).

For an intra-period dW₁(15) and dS₁(3)

$${w}_{dir}^{′} = {w}_{dir} ∓ τ {dW}_{1}$$

$${w}_{spd}^{′} = {w}_{spd} ∓ τ {dS}_{1}$$

Inter-period dW₂(45) and dS₂(6)

$${w}_{dir}^{′} = {w}_{dir} ∓ τ {dW}_{2}$$

$${w}_{spd}^{′} = {w}_{spd} ∓ τ {dS}_{2}$$

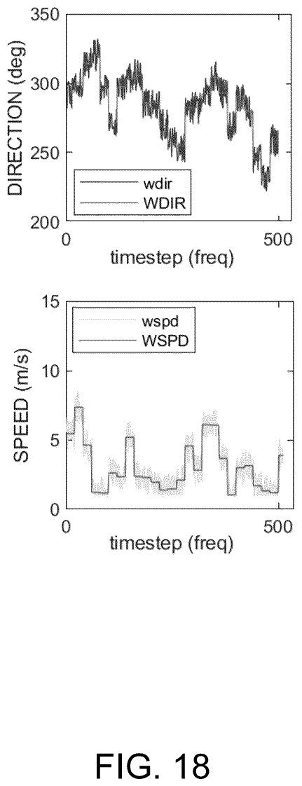

As illustrated in FIG. 18, direction of wind (in degrees) versus timestep (freq) and speed versus timestep (freq) are illustrated. For the synthetic wind model a set number of periods is set. nₚ=(60 T)/t. An initialization is also performed where the starting conditions may be randomly set:

$${w}_{DIR}^{j = 1} = {w}_{DIR}^{0} and {w}_{SPD}^{j = 1} = {w}_{SPD}^{0}$$

$$For j = 1 : {n}_{p}$$

$$For k = 1 : t$$

$${w}_{dir}^{k} = {w}_{DIR}^{j} ∓ τ {dW}_{1}$$

$${w}_{spd}^{k} = {w}_{SPD}^{j} ∓ τ {dS}_{1}$$

$$Store : [ {w}_{DIR}^{j} {W}_{SPD}^{j} {w}_{dir}^{k} {W}_{spd}^{k} ]$$

$$k = k + 1$$

$${w}_{DIR}^{j + 1} = {w}_{DIR}^{j} ∓ τ {dW}_{2}$$

$${w}_{SPD}^{j + 1} = {w}_{SPD}^{j} ∓ τ {dS}_{2}$$

$$j = j + 1.$$

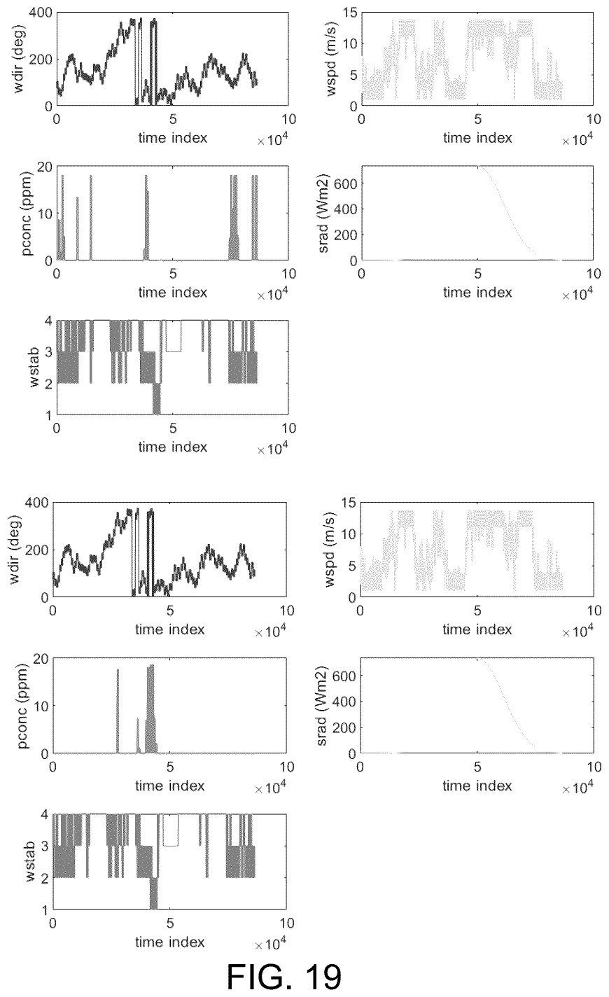

For FIG. 19, the synthetic wind model, the solar radiation profile and wind stability condition is established over period T. An expected concentration reading, in parts per million, is established for a given sensor using the forward model: Gaussian plume model (GPM) or other. The desired number of wind realizations n_{w} is then repeated. The number of wind realizations is given as n_{w}.

As stated above, the sensor location problem demands that the sensors are placed within site constraints, but outside of the constrained sub-space region. The source inversion problem demands that the leak source is restricted to the interior of the sub-space regions. Thus, this procedure helps to optimally place sensors and isolate leak sources subject to sub-space restrictions. This can help improve the search to only feasible locales and assists in direct source attribution (to the sub-space of interest).

Example embodiments of the claims are disclosed. The example embodiments should not be considered limiting. In one example embodiment, a method to monitor emissions from a site is disclosed. The method may comprise designating a perimeter for the site to be monitored. The method may also comprise predefining locations for a number of sensors and locations for the sensors along the perimeter of the site. The method may also comprise defining a wind realization model for the site. The method may also comprise defining a set of candidate leak points for the site. The method may also comprise using a binary optimization method to select a first sub-set of sensor locations for evaluation of monitoring of the emissions to achieve a result. The method may also comprise further using the binary optimization method to select at least a second sub-set of sensor locations for evaluating the emissions to achieve a second result. The method may also comprise comparing the first result with the second result. The method may also comprise choosing either the first sub-set of sensor locations or the second sub-set of sensor locations based upon a factor defined by an evaluator.

In another example embodiment, the method may be performed wherein the emissions are methane based.

In another example embodiment, the method may be performed wherein the wind realization model incorporates environmental factors.

In another example embodiment, the method may be performed wherein the wind realization model incorporates at least one of solar radiation, ground-based structures, and historical weather patterns.

In another example embodiment, the method may be performed wherein the candidate leak points for the site are based upon field locations for gaseous emissions.

In another example embodiment, the method may be performed wherein a spacing between sensors is defined by a minimum separation distance.

In another example embodiment, the method may be performed wherein the factor chosen by the evaluator is based on at least one of an economic cost, a geographic location, and an emission monitoring value.

In another example embodiment, the method may be performed further comprising defining a location sub-set of positions where sensors are not restricted from being placed and excluding the sub-set of positions from possible sensor locations.

In another example embodiment, the method may be performed wherein the binary optimization method uses advanced meta-heuristics.

In another example embodiment, a method for monitoring a leak from a leak source is disclosed. The method may comprise designating a perimeter for the site to be monitored. The method may also comprise identifying a number of sub-spaces within the perimeter in which a leak source may occur, each of the sub-spaces having a bounded region with a bounded perimeter. The method may also comprise identifying within each of the number of sub-spaces a location for a potential leak source within an interior of each sub-space. The method may also comprise locating at least one sensor to be located for monitoring each sub-space for each potential leak source, wherein the locating is done exterior to each bounded perimeter to achieve a first sub-set of sensor locations. The method may also comprise defining a wind realization model for the site. The method may also comprise calculating a center of mass for the site from each of the sensors located for monitoring. The method may also comprise using an optimization method that uses the center of mass wherein a penalty is assigned for each sensor placed within a subspace, the optimization method providing a first result. The method may also comprise further using the optimization method to select at least a second sub-set of sensor locations in a second subspace to achieve a second result, wherein the penalty is assigned for each sensor placed within the second subspace. The method may also comprise comparing the first result with the second result. The method may also comprise choosing either the first sub-set of sensor locations or the second sub-set of sensor locations based upon at least one of the first result, the second result and a factor defined by an evaluator. In embodiments, the perimeter does not have to be a complex polygon. In some embodiments, the problem may be convex for interior or exterior placement.

In another example embodiment, the method may be performed wherein the sub-spaces are defined in two-dimensions.

In another example embodiment, the method may be performed wherein the sub-spaces are defined in three dimensions.

In another example embodiment, the method may be performed wherein the sub-spaces are defined by a segmentation procedure. This entails the use of AI/ML to automatically aid the extraction of pertinent data from an image of the client site.

In another example embodiment, the method may be performed wherein the choosing of either the first sub-set of sensor locations or the second sub-set of sensor locations is based on a feasibility.

In another example embodiment, the method may be performed wherein the feasibility is based upon exclusion of sensors from defined sub-spaces.

In another example embodiment, the method may be performed wherein the wind model uses at least one of historical data from the site or present wind conditions from the site.

In another example embodiment, the method may be performed wherein the factor defined by the evaluator is based upon at least one of a monitoring resolution for the leak, a number of sensors to be located at the site, and an overall economic cost of installing sensors at the site.

In another example embodiment, the method may be performed wherein an inversion procedure is used for calculating results.

In another example embodiment, the method may be performed wherein input data for the inversion procedure includes wind data, sensor data, solar data, and sensor location.

In another example embodiment, a method for modeling a potential leak from a leak source at a site is disclosed. The method may comprise designating a perimeter for the site to be monitored. The method may also comprise identifying a number of sub-spaces within the perimeter in which a leak source may occur, each of the sub-spaces having a bounded region with a bounded perimeter. The method may also comprise identifying within each of the number of sub-spaces a location for a potential leak source within an interior of each sub-space. The method may also comprise locating at least one sensor to be located for monitoring each sub-space for each potential leak source, wherein the locating is done exterior to each bounded perimeter to achieve a first sub-set of sensor locations. The method may also comprise defining a wind realization model for the site. The method may also comprise defining a sensor model for the site. The method may also comprise defining a leak source model for the site. The method may also comprise calculating a wind realization model result, a sensor model result, and a leak source model result, and supplying each of the wind realization model result, sensor model result, and the leak source model result into an inversion procedure. The method may also comprise using the inversion procedure, developing a detection coverage map for the site.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may be varied in many ways. Such variations are not to be regarded as a departure from the disclosure, and all such modifications are intended to be included within the scope of the disclosure.

While embodiments have been described herein, those skilled in the art, having benefit of this disclosure, will appreciate that other embodiments are envisioned that do not depart from the inventive scope. Accordingly, the scope of the present claims or any subsequent claims shall not be unduly limited by the description of the embodiments described herein.

What is claimed is:

1. A method to monitor emissions from a site, comprising:
designating a perimeter for the site to be monitored;
predefining locations for a number of sensors and locations for the sensors along the perimeter of the site;
defining a wind realization model for the site;
defining a set of candidate leak points for the site;
using a binary optimization method to select a first sub-set of sensor locations for evaluation of monitoring of the emissions to achieve a result;
further using the binary optimization method to select at least a second sub-set of sensor locations for evaluating the emissions to achieve a second result;
comparing the first result with the second result; and
choosing either the first sub-set of sensor locations or the second sub-set of sensor locations based upon a factor defined by an evaluator.

2. The method according to claim 1, wherein the emissions are methane based.

3. The method according to claim 1, wherein the wind realization model incorporates environmental factors.

4. The method according to claim 3, wherein the wind realization model incorporates at least one of solar radiation, ground-based structures, and historical weather patterns.

5. The method according to claim 1, wherein the candidate leak points for the site are based upon field locations for gaseous emissions.

6. The method according to claim 1, wherein a spacing between sensors is defined by a minimum separation distance.

7. The method according to claim 1, wherein the factor chosen by the evaluator is based on at least one of an economic cost, a geographic location, and an emission monitoring value.

8. The method according to claim 1, further comprising defining a location sub-set of positions where sensors are not restricted from being placed and excluding the sub-set of positions from possible sensor locations.

9. The method according to claim 1, wherein the binary optimization method uses advanced meta-heuristics.

10. A method for monitoring a leak from a leak source, comprising:
designating a perimeter for the site to be monitored;
identifying a number of sub-spaces within the perimeter in which a leak source may occur, each of the sub-spaces having a bounded region with a bounded perimeter;
identifying within each of the number of sub-spaces a location for a potential leak source within an interior of each of the sub-spaces;
locating at least one sensor to be located for monitoring each of the sub-spaces for each potential leak source, wherein the locating is done exterior to each bounded perimeter to achieve a first sub-set of sensor locations;
defining a wind realization model for the site;
calculating a center of mass for the site from each of the sensors located for monitoring;
using an optimization method that uses the center of mass of each of the subspaces to assess feasibility for each of the sensor placed within a subspace, the optimization method providing a first result;
further using the optimization method to select at least a second sub-set of sensor locations in a second subspace to achieve a second result, wherein the penalty is assigned for each sensor placed within the second subspace;
comparing the first result with the second result; and
choosing either the first sub-set of sensor locations or the second sub-set of sensor locations based upon at least one of the first result, the second result and a factor defined by an evaluator.

11. The method according to claim 10, wherein the sub-spaces are defined in two-dimensions.

12. The method according to claim 10, wherein the sub-spaces are defined in three dimensions.

13. The method according to claim 10, wherein the sub-spaces are defined by a segmentation procedure.

14. The method according to claim 10, wherein the choosing of either the first sub-set of sensor locations or the second sub-set of sensor locations is based on a feasibility.

15. The method according to claim 14, wherein the feasibility is based upon exclusion of sensors from defined sub-spaces.

16. The method according to claim 10, wherein the wind model uses at least one of historical data from the site or present wind conditions from the site.

17. The method according to claim 10, wherein the factor defined by the evaluator is based upon at least one of a monitoring resolution for the leak, a number of sensors to be located at the site, and an overall economic cost of installing sensors at the site.

18. The method according to claim 10, wherein an inversion procedure is used for calculating results.

19. The method according to claim 18, wherein input data for the inversion procedure includes wind data, sensor data, solar data, and sensor location.

20. A method for modeling a potential leak from a leak source at a site, comprising:
designating a perimeter for the site to be monitored;
identifying a number of sub-spaces within the perimeter in which a leak source may occur, each of the sub-spaces having a bounded region with a bounded perimeter;
identifying within each of the number of sub-spaces a location for a potential leak source within an interior of each sub-space;
locating at least one sensor to be located for monitoring each sub-space for each potential leak source, wherein the locating is done exterior to each bounded perimeter to achieve a first sub-set of sensor locations;
defining a wind realization model for the site;
defining a sensor model for the site;
defining a leak source model for the site;
calculating a wind realization model result, a sensor model result, and a leak source model result, and supplying each of the wind realization model result, sensor model result, and the leak source model result into an inversion procedure; and
using the inversion procedure, developing a detection coverage map for the site.
